(19) United States
(12) Patent Application Publication
(10) Pub. No.: US 2026/0330602 A1
(43) Pub. Date: Oct. 22, 2026

(54) MACHINE LEARNING SYSTEMS AND RELATED ASPECTS FOR THREE MATERIAL DECOMPOSITION USING A TRIPLE-LAYER FLAT-PANEL DETECTOR

(71) Applicant: THE JOHNS HOPKINS UNIVERSITY, Baltimore, MD (US)

(72) Inventors: Jianan GANG, Baltimore, MD (US); Joseph Webster STAYMAN, Baltimore, MD (US); Xiao JIANG, Baltimore, MD (US)

(21) Appl. No.: 19/119,307

(22) PCT Filed: Oct. 18, 2023
(86) PCT No.: PCT/US2023/035371

§ 371 Date: Apr. 8, 2025

Related U.S. Application Data
(60) Provisional application No. 63/379,944, filed on Oct. 18, 2022.

(51) Int. Cl. G06T 5/50; A61B 6/00; A61B 6/42; G06T 5/60

(52) U.S. Cl. CPC G06T 5/50; A61B 6/4208; A61B 6/481; A61B 6/482; G06T 5/60; G06T 2207/10121; G06T 2207/20081; G06T 2207/20084; G06T 2207/30008

(57) ABSTRACT

Provided herein are computer-implemented methods of decomposing image data obtained from a subject. In some embodiments, the methods include receiving, by a computer, the image data from a triple-layer flat-panel detector (TL-FPD) that comprises at least three energy channels and separating, by the computer, the image data into at least three component materials that comprise calcium, water, and contrast agent. Related systems, computer readable media, and additional methods are also provided.

### CROSS-REFERENCE TO RELATED APPLICATIONS

This application is the national stage entry of International Patent Application No. PCT/US2023/035371, filed on Oct. 18, 2023, and published as WO 2024/086197 A1 on Apr. 25, 2024, which claims the benefit of U.S. Provisional Patent Application Ser. No. 63/379,944, filed on Oct. 18, 2022, which are hereby incorporated by reference herein in their entireties.

### STATEMENT REGARDING FEDERALLY SPONSORED RESEARCH OR DEVELOPMENT

This invention was made with government support under contract EB030494 awarded by the National Institutes of Health. The government has certain rights in the invention.

### BACKGROUND

X-ray dual-energy (DE) imaging provides capabilities for material quantification and non-contrast images synthesis. Recently, DE imaging with flat-panel detectors (FPDs) has been explored and shown promising application in many 2D imaging scenarios. For example, DE chest radiography improves the diagnosis of pulmonary nodules and small lung tumors, and in the detection of cardiovascular diseases. DE imaging is also being explored in interventional radiology—providing improved iodine visualization for hemorrhage detection, assessments after revascularization, and improved vascular lumen depiction.

Several methods have been developed for spectral radiography/interventional data acquisition. Tube voltage switching is one form of spectral acquisition which is compatible with conventional single-channel detector systems. However, this utilizes multiple acquisitions and the potential for mismatched projections in the presence of the patient motion. In contrast, multi-layer flat-panel detectors can provide simultaneous projections that minimize motion-induced artifacts. Dual-layer flat-panel detector (DLFPD) has been explored for radiography and interventional imaging systems. Projections acquired by DL-FPD permit differentiation between two materials, typically soft tissues and bone. A large number diagnostic and interventional application involves the administration of iodine contrast agent and would benefit from further separation of iodine and calcium. Such separation is challenging with DL-FPD. Current methods for contrast visualization include digital subtraction angiography (DSA) where a pre-contrast projection is employed to remove background anatomy. Any movement between the pre-contrast scan and post-contrast imaging leads to artifacts in the contrast-only image. Thus, DSA is generally not applicable to cardiac imaging and other targets where there is significant motion.

Accordingly, there is a need for additional methods of decomposing image data.

### SUMMARY

The present disclosure provides, in certain aspects, an artificial intelligence (AI) system and related methods capable of decomposing image data (e.g., into three materials, such as bone, water, and an iodinated contrast agent) from a triple-layer flat-panel detectors (TL-FPDs) that comprise at least three energy channels. The methods, systems, and other aspects of the present disclosure typically use machine learning models to perform this decomposition. The ability to directly decompose, for example, radiographic/flouroscopic data into water, bone, and contrast agent channels is extremely useful for medical diagnostics and interventions. Classic techniques for iodine visualization include digital subtraction angiography where a pre-contrast image is subtracted from following images to show only the iodine. This technique is sensitive to motion and cannot be used in all circumstances (e.g. cardiac imaging where there is constant motion). In contrast, the “one-shot” techniques disclosed herein permit direct visualization of any of the three channels (bone, water, iodine) and is insensitive to motion. These and other aspects will be apparent upon a complete review of the present disclosure, including the accompanying figures.

According to various embodiments, a method of decomposing image data obtained from a subject using a computer is presented. The method includes: receiving, by the computer, the image data from a triple-layer flat-panel detector (TL-FPD) that comprises at least three energy channels; and, separating, by the computer, the image data into at least three component materials that comprise calcium, water, and contrast agent, thereby decomposing the image data obtained from the subject using the computer.

Various optional features of the above embodiments include the following. The contrast agent comprises an iodinated contrast agent. The image data comprises radiographic and/or fluoroscopic image data. The method is substantially insensitive to motion of and/or in the subject. The method provides direct visualization of any of the three energy channels. The image data comprises interventional radiographic image data. The separating step comprises using at least one processing technique. The processing technique comprises a polynomial-based technique. The processing technique comprises a model-based technique. The processing technique comprises a machine learning technique.

According to various embodiments, a system for decomposing image data obtained from a subject is presented. The system includes a processor; and a memory communicatively coupled to the processor, the memory storing instructions which, when executed on the processor, perform operations including: receiving the image data from a triple-layer flat-panel detector (TL-FPD) that comprises at least three energy channels; and, separating the image data into at least three component materials that comprise calcium, water, and contrast agent.

Various optional features of the above embodiments include the following. The contrast agent comprises an iodinated contrast agent. The image data comprises radiographic and/or fluoroscopic image data. The image data comprises interventional radiographic image data. The processor separates the image data into the at least three component materials that comprise calcium, water, and contrast agent using at least one processing technique. The processing technique comprises a polynomial-based technique. The processing technique comprises a model-based technique. The processing technique comprises a machine learning technique.

According to various embodiments, a computer readable media is presented. The computer readable media comprises non-transitory computer executable instructions which, when executed by at least one electronic processor, perform at least: receiving image data from a triple-layer flat-panel detector (TL-FPD) that comprises at least three energy channels; and, separating the image data into at least three component materials that comprise calcium, water, and contrast agent.

Various optional features of the above embodiments include the following. The contrast agent comprises an iodinated contrast agent. The image data comprises radiographic and/or fluoroscopic image data. The image data comprises interventional radiographic image data. The electronic processor separates the image data into the at least three component materials that comprise calcium, water, and contrast agent using at least one processing technique. The processing technique comprises a polynomial-based technique. The processing technique comprises a model-based technique. The processing technique comprises a machine learning technique.

### DRAWINGS

The above and/or other aspects and advantages will become more apparent and more readily appreciated from the following detailed description of examples, taken in conjunction with the accompanying drawings, in which:

FIG. 1 is a flow chart that schematically shows exemplary method steps of decomposing image data obtained from a subject according to some aspects disclosed herein.

FIG. 2 is a schematic diagram of an exemplary system suitable for use with certain aspects disclosed herein.

FIGS. 3A and B are plots that show the spectral sensitivities and spatial resolution, respectively, of a simulated TL-FPD.

FIG. 4 depicts an exemplary ResUnet network architecture. This network transforms the log-normalized triple-layer projections to material density projections. The encoder path (ResBlock1~ResBlock5) maps the input 720×720×3 images to a 45×45×512 high-dimensional representation, and the decoder path (ResBlock6~ResBlock9) recovers the images to the original size.

FIG. 5 shows projection-domain material decomposition (rows 1, 3, and 5) using polynomial-based, model-based and learning-based methods. The remaining images represent the absolute error maps, and the box indicates the areas used for bias and TV calculation. Quantitative results are listed at the top-left corners (unit: mg/mm²).

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms (“a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Electronic neural network: As used herein, “electronic neural network” or “neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., peptide sequence and binding value pair data sets or the like).

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial or electronic neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., a known pathology, such as melanoma and/or the like).

System: As used herein, “system” in the context of analytical instrumentation refers a group of objects and/or devices that form a network for performing a desired objective.

Value: As used herein, “value” generally refers to an entry in a data set that can be anything that characterizes the feature to which the value refers. This includes, without limitation, numbers, words or phrases, symbols (e.g., + or −) or degrees.

### DESCRIPTION OF THE EMBODIMENTS

Reference will now be made in detail to example implementations. These embodiments are described in sufficient detail to enable those skilled in the art to practice the invention and it is to be understood that other embodiments may be utilized and that changes may be made without departing from the scope of the invention. The following description is, therefore, merely exemplary.

### I. INTRODUCTION

X-ray spectral imaging has been increasingly investigated in radiography and interventional imaging. Flat-panel detectors with more than one detection layer have demonstrated advantages in providing separate soft tissue and bone images. Current dual-layer flat-panel detectors (DL-FPD) have limited capability to further differentiate between iodinated contrast agent and bony/calcified structures. In aspects of the present disclosure, we use a triple-layer flat-panel detector (TL-FPD) and demonstrate the feasibility of three-material (e.g., water/calcium/iodine) decomposition. A physical model of TL-FPD, including system geometry, spectrum sensitivities, blur and noise models is also disclosed. Using simulated triple-layer projections, three-material decompositions were performed using three different processing methods: polynomial-based, model-based, and a machine learning-based method (ResUnet). As illustrated in an Example provided in the present disclosure, we found that the polynomial-based method leads to very noisy images with poor differentiation between calcium and iodine maps. The model-based method achieved much lower noise levels than the polynomial-based method but exhibited residual errors between the iodine and calcium channels. The ResUnet method offered the best decompositions among the investigated methods in terms of root mean square error from the ground truth and noise in the material maps. These exemplary results demonstrate the feasibility of three-material decomposition using TL-FPD and suggest a path for clinical translation of single-shot contrast/iodine differentiation in radiography and fluoroscopy.

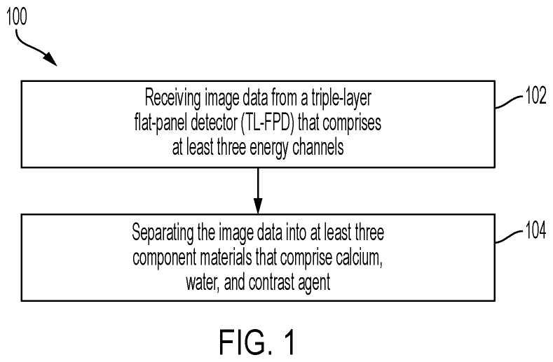

To illustrate, FIG. 1 is a flow chart that schematically shows exemplary method steps of decomposing image data obtained from a subject according to some aspects disclosed herein. As shown, method 100 includes receiving, by a computer, the image data from a triple-layer flat-panel detector (TL-FPD) that comprises at least three energy channels (step 102). In addition, method 100 also includes separating, by the computer, the image data into at least three component materials that comprise calcium, water, and contrast agent to thereby decompose the image data obtained from the subject using the computer (step 104).

In some embodiments, the contrast agent comprises an iodinated contrast agent. In some embodiments, the image data comprises radiographic and/or fluoroscopic image data. In some embodiments, method 100 is substantially insensitive to motion of and/or in the subject. In some embodiments, method 100 provides direct visualization of any of the three energy channels. In some embodiments, the image data comprises interventional radiographic image data. In some embodiments, the separating step comprises using at least one processing technique (e.g., a polynomial-based technique, a model-based technique, and/or a machine learning technique).

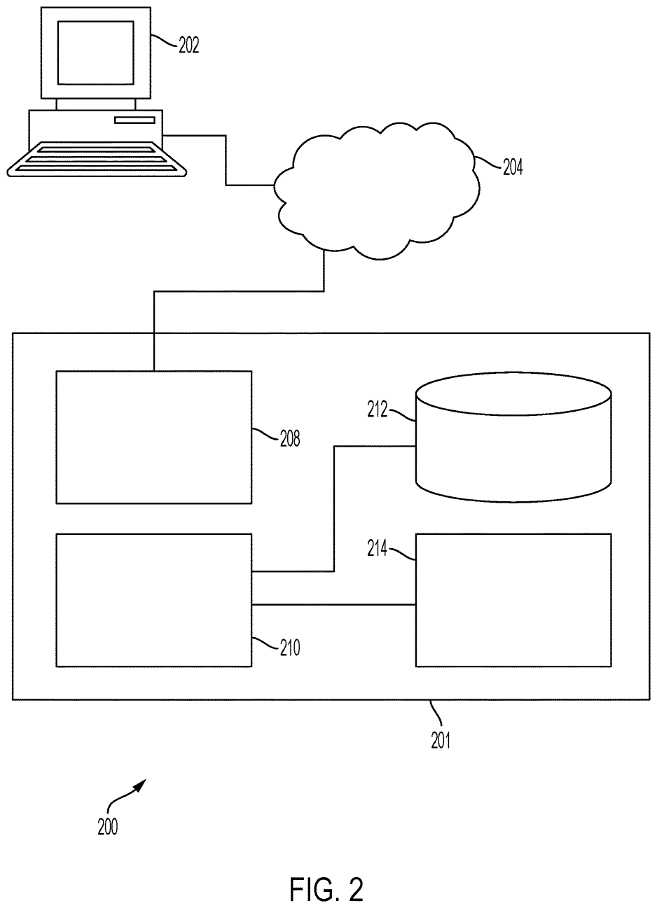

FIG. 2 is a schematic diagram of a hardware computer system 200 suitable for implementing various embodiments. For example, FIG. 2 illustrates various hardware, software, and other resources that can be used in implementations of any of methods disclosed herein, including method 100 and/or one or more instances of an electronic neural network. System 200 includes training corpus source 202 and computer 201. Training corpus source 202 and computer 201 may be communicatively coupled by way of one or more networks 204, e.g., the internet.

Training corpus source 202 may include an electronic clinical records system, such as an LIS, a database, a compendium of clinical data, or any other source of image data suitable for use as a training corpus as disclosed herein. According to some embodiments, each component is implemented as a vector, such as a feature vector, that represents a respective tile. Thus, the term “component” refers to both a tile and a feature vector representing a tile.

Computer 201 may be implemented as any of a desktop computer, a laptop computer, can be incorporated in one or more servers, clusters, or other computers or hardware resources, or can be implemented using cloud-based resources. Computer 201 includes volatile memory 214 and persistent memory 212, the latter of which can store computer-readable instructions, that, when executed by electronic processor 210, configure computer 201 to perform any of the methods disclosed herein, including method 100, and/or form or store any electronic neural network, and/or perform any classification technique as described herein. Computer 201 further includes network interface 208, which communicatively couples computer 201 to training corpus source 202 via network 204. Other configurations of system 200, associated network connections, and other hardware, software, and service resources are possible.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

### II. DESCRIPTION OF EXAMPLE EMBODIMENTS

### Example: Approaches for Three Material Decomposition Using a Triple-Layer Flat-Panel Detector

### 1. Introduction

In this example, we explore the ability of a triple-layer flat-panel detector (TL-FPD) to obtain water, calcium, and iodine images in a single acquisition—avoiding the motion artifacts of DSA and providing additional capability over DL-FPDs. We emulate a TL-FPD based on an existing Varex DL-FPD architecture. We investigate the feasibility of applying single-shot TL-FPD and three-material decomposition using three different processing techniques (polynomial-based, model-based, and machine learning) and compare the numerical accuracy and noise levels for each method.

### 2. Methods

### 2.1 Triple-Layer Flat-Panel Radiography Simulation

The TL-FPD modeled in this example is based on a prototype DL-FPD consisting of two stacked amorphous silicon indirect detectors with an interstitial copper filter. In the triple layer simulation, we retained the top and bottom layers (with CsI layers of 200 μm and 550 μm, respectively) but replaced the copper filter with another detector layer (400 μm CsI). We presumed the pixel grids of three layers are perfectly aligned, and each had 720×720 pixels with pixel size of 0.6 mm×0.6 mm. The source-to-axis distance was 828 mm, and the source-to-detector distances were 1126 mm, 1129 mm, 1132 mm for each of the three layers, respectively.

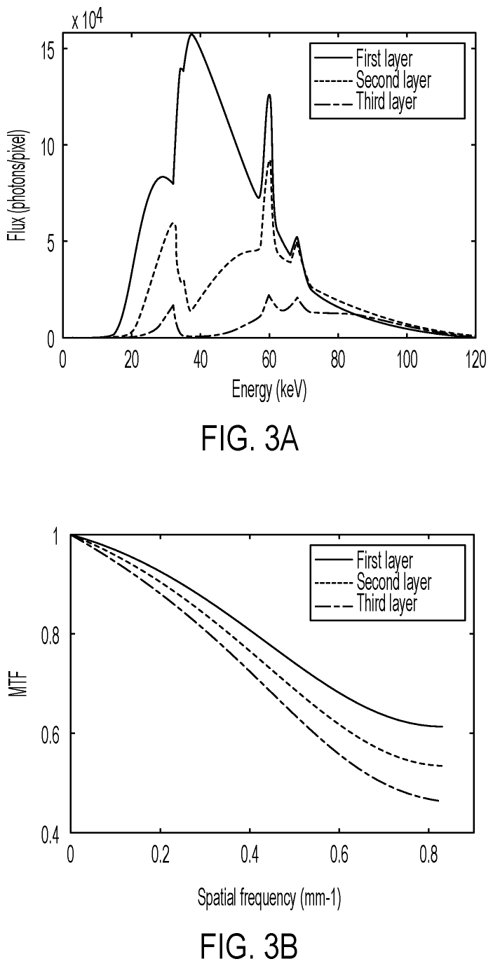

Projection images are simulated using the following forward model:

$$\overline{{y}_{l}} = {B}_{l} {S}_{l} exp ( - Q q ) , l = 1 , 2 , 3$$

where l represents the lth detection layer, Q is the matrix of mass attenuation coefficients for each basis materials, and q represents the line-integrals of basis material densities. As described in Wang et al. (“High-resolution model-based material decomposition in dual-layer flat-panel CBCT,” Med Phys 48(10), 6375-6387 (2021)), the total spectral sensitivity Sₗ models the combined effects of incident spectrum and spectral sensitivity of each detector layer. The incident spectrum is generated by Spektr at 120 kVp and 10 mAs. Detector sensitivity is computed using the quantum detection efficiency of CsI. Detector blur of the top and bottom layers (B₁, B₃) were measured in terms of the modulation transfer function (MTF) in previous work. The MTF of the middle layer is estimated by interpolating the top and bottom layer MTFs based on scintillator thickness. The total spectral sensitivity and MTF curves are shown in FIG. 3. In addition, independent Poisson noise is added to projection data prior to applying detector blur.

### 2.2 Polynomial-Based Material Decomposition

Theoretically, TL-FPD has the capability of discriminating three materials because of the three energy channels. However, the projection-domain material decomposition is an ill-posed inverse problem with no explicit solution. The polynomial-based method approximates the desired material density projections using the polynomials with respect to the log-normalized projections using the following polynomial mapping and coefficients optimized according to the given error function:

$${\hat{q}}_{m} = {\sum }_{n = 1}^{N} {\sum }_{i + j + k = n} {α}_{m , ijk} {p}_{1}^{i} {p}_{2}^{j} {p}_{3}^{k} , m = 1 , 2 , … M$$

$$err = {‖ {\sum }_{n = 1}^{N} {\sum }_{i + j + k = n} {α}_{m , ijk} {p}_{1}^{i} {p}_{2}^{j} {p}_{3}^{k} - {q}_{m} ‖}_{2}^{2}$$

where \hat{q}ₘ is the estimated line-integral of the mth basis material density and M is the number of basis materials; pₗ represents the log-transformed projections from the lth layer. The highest order polynomial used in this example is N=4. With the assumption that the polynomial coefficients α_{l,ijk} only depends on the spectral sensitivities and the basis materials, α_{l,ijk} is calibrated using known-composition phantoms by minimizing the above error function.

The three detection layers exhibit different magnifications and blur models, which hampers the pixel-by-pixel material decomposition. To mitigate this effect, the second- and third-layer projections were registered to the first layer via cubic interpolation. Projection deblurring was then performed via dividing the raw projection by the 2-dimension MTF in the frequency domain.

### 2.3 Model-Based Material Decomposition

Model-based decomposition makes full use of the spectral information and the noise characteristics. Assume the detector measurements follow independent multivariate Gaussian distribution with mean yₗ and covariance Σₗ=D[yₗ], the material density integrals were estimated using the following regularized likelihood objective function:

$$Φ ( q ) = {\sum }_{l = 1}^{3} {( {y}_{l} - {S}_{l} exp ( - Qq ) )}^{T} {Σ}_{l}^{- 1} ( {y}_{l} - {S}_{l} exp ( - Q q ) ) + R ( q )$$

where yₗ is the vectorized measurements from the lth layer, R is a quadratic regularization term that penalizes the differences in the 4-nearest neighbor pixels within each material map. Each material map has its own regularization strength βₘ which was empirically set to 1, 10, 500 for the water, calcium, and iodine basis, respectively. Note that the data fidelity term is naturally separable for each pixel, and the regularization may be replaced by a separable surrogate. We therefore adopted the Separable Paraboloidal Surrogates (SPS) algorithm for optimization, using the polynomial-based results as the initial guess.

### 2.4 Learning-Based Material Decomposition

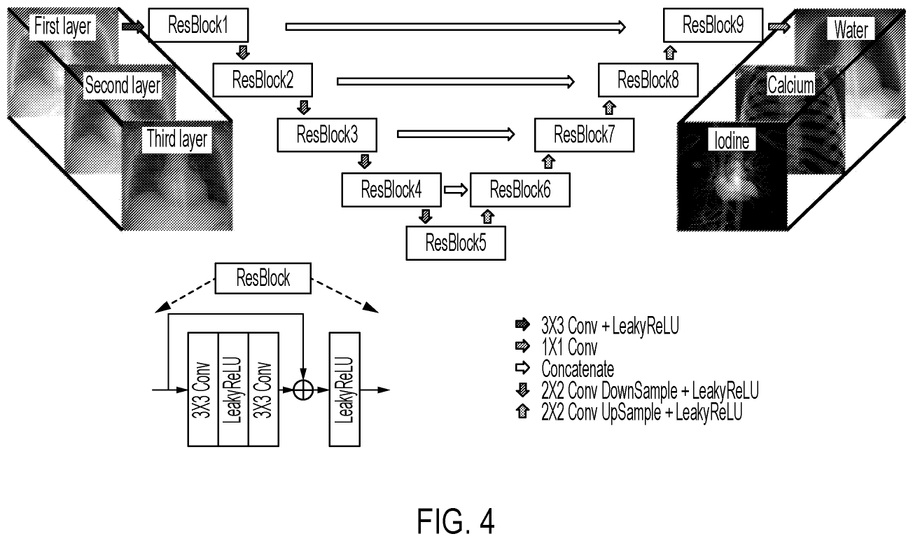

Deep learning has been widely used in medical imaging tasks. In this study, we applied the Residual U-shape Network (ResUnet) to perform material decomposition of data acquired from the TL-FPD. The ResUnet, as shown in FIG. 4, contains 9 residual blocks (ResBlock). Each block consists of two 3×3 convolution layers followed by a LeakyReLU activation. Residual connection which directly maps the block input to output was applied in each block to mitigate the gradient vanishing and explosion problems in deep networks. The three projections from the TL-FPD were concatenated to form a three-channel input to the network. A 3×3 convolution is firstly applied to increase the number of channels to 32. The first five ResBlocks forms the encoder path to map the input data to high-dimension space. After each encoder block, the image dimension reduced to half, while the feature channels doubled. The last four ResBlocks forms the decoder path which is connected to the encoder path by concatenation. Each decoder block increases the image dimension while reduces features channels. Finally, a 1×1 convolution is applied to reduce the channels to the number of basis materials. The network yields water, calcium, and iodine density line integrals as the output.

We generated 54 realizations of digital chest phantoms using the XCAT model. Each phantom had 384×384×384 voxels with 1 mm cubic voxels. To obtain the physical densities for each basis materials, non-contrast attenuation images were generated at 60 keV and 100 keV, followed by an image-domain decomposition to obtain the water and calcium densities. Iodine maps were based on vessel and cardiac elements of XCAT and randomly assigned a concentration ranging from 8 mg/ml to 12 mg/ml. For each phantom, 72 projections were simulated over 360° with uniform angular sampling. Training, validation, and testing dataset contains projections from 38, 10, and 6 phantoms, respectively (with no repetition of phantoms between sets). The ResUnet was implemented using PyTorch, and the network parameters were optimized by minimizing the L1 distance between the output and the label material projections. The network training used the Adam optimizer with a batch size of 4 and terminated after 50 epochs based on an empirical evaluation of the loss function.

### 2.5 Evaluation

The performance of different decomposition methods was evaluated and compared in terms of the numerical accuracy and the noise level. Root-Mean-Square Error (RMSE) was computed for each basis image to quantify the overall decomposition accuracy. To further investigate the accuracy of calcium-iodine separation, a region-of-interest (ROI) with relatively uniform and overlapping calcium and iodine was selected. Bias within the ROI was computed as the mean value difference between decomposition results and the ground truth. Since the area was not perfectly uniform, to estimate noise levels we computed the total variation (TV) of the selected ROI.

### 3. RESULTS

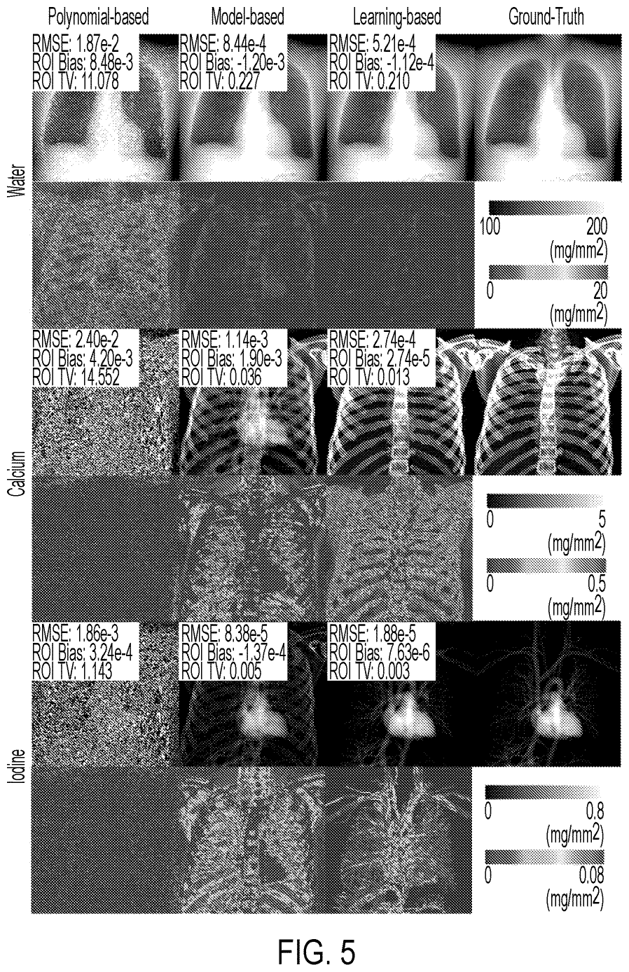

Material decomposition results are shown in FIG. 5, with quantitative evaluation metrics listed in the top-left corners. Based on the RMSE values, Deep learning decomposition achieved significantly lower RMSE over other two methods. Polynomial-based results were severely corrupted by noise. In comparison, model-based decomposition results have much lower noise, permitting visualization of finer details within the lungs and lower ROI TV in the calcium and iodine images. However, calcium and iodine cannot be perfectly separated, as evidenced by the appearance of both the spine and heart in both calcium and iodine images. Compared with the model-based method, deep learning was able to reduce noise and bias in the calcium and iodine images. With the accurate calcium/iodine decomposition, the vascular system was effectively separated from the background by the ResUnet, and the error image indicates that the intensity of large arteries as well as the vessel trees shows high consistency with the ground truth.

### 4. CONCLUSION

In this example, we investigated the potential for three material decomposition using a flat-panel detector with triple-layer configuration. Since the spectral sensitivities of three layers have large overlaps, and the energy dependence of calcium and iodine attenuation are similar, accurate three-material decomposition is very challenging for the triple-layer radiography system. Three decomposition algorithms were implemented and evaluated. Initial results suggested that polynomial-based method suffers very high noise levels; the model-based method has better noise performance but is unable to perfectly separate iodine and calcium. A learning-based method (ResUnet) outperforms other two methods on both material separation and calcium/iodine quantification.

This example shows the significant potential for three-material decomposition using TL-FPDs. Such technology enables motion-free angiography (for difficult targets like cardiac fluoroscopy) and other spectral x-ray applications. We note that the ResUnet results exhibit blur on the small vessels and spine edges. Optimization of the neural net architecture and/or loss function enables better spatial resolution. The model can also be generalized to different noise levels and over larger ensembles of evaluation data.

Some further aspects are defined in the following clauses:

Clause 1: A method of decomposing image data obtained from a subject using a computer, the method comprising: receiving, by the computer, the image data from a triple-layer flat-panel detector (TL-FPD) that comprises at least three energy channels; and, separating, by the computer, the image data into at least three component materials that comprise calcium, water, and contrast agent, thereby decomposing the image data obtained from the subject using the computer.

Clause 2: The method of Clause 1, wherein the contrast agent comprises an iodinated contrast agent.

Clause 3: The method of Clause 1 or Clause 2, wherein the image data comprises radiographic and/or fluoroscopic image data.

Clause 4: The method of any one of the preceding Clauses 1-3, wherein the method is substantially insensitive to motion of and/or in the subject.

Clause 5: The method of any one of the preceding Clauses 1-4, wherein the method provides direct visualization of any of the three energy channels.

Clause 6: The method of any one of the preceding Clauses 1-5, wherein the image data comprises interventional radiographic image data.

Clause 7: The method of any one of the preceding Clauses 1-6, wherein the separating step comprises using at least one processing technique.

Clause 8: The method of any one of the preceding Clauses 1-7, wherein the processing technique comprises a polynomial-based technique.

Clause 9: The method of any one of the preceding Clauses 1-8, wherein the processing technique comprises a model-based technique.

Clause 10: The method of any one of the preceding Clauses 1-9, wherein the processing technique comprises a machine learning technique.

Clause 11: A system for decomposing image data obtained from a subject, the system comprising: a processor; and a memory communicatively coupled to the processor, the memory storing instructions which, when executed on the processor, perform operations comprising: receiving the image data from a triple-layer flat-panel detector (TL-FPD) that comprises at least three energy channels; and, separating the image data into at least three component materials that comprise calcium, water, and contrast agent.

Clause 12: The system of Clause 11, wherein the contrast agent comprises an iodinated contrast agent.

Clause 13: The system of Clause 11 or Clause 12, wherein the image data comprises radiographic and/or fluoroscopic image data.

Clause 14: The system of any one of the preceding Clauses 11-13, wherein the image data comprises interventional radiographic image data.

Clause 15: The system of any one of the preceding Clauses 11-14, wherein the processor separates the image data into the at least three component materials that comprise calcium, water, and contrast agent using at least one processing technique.

Clause 16: The system of any one of the preceding Clauses 11-15, wherein the processing technique comprises a polynomial-based technique.

Clause 17: The system of any one of the preceding Clauses 11-16, wherein the processing technique comprises a model-based technique.

Clause 18: The system of any one of the preceding Clauses 11-17, wherein the processing technique comprises a machine learning technique.

Clause 19: A computer readable media comprising non-transitory computer executable instructions which, when executed by at least one electronic processor, perform at least: receiving image data from a triple-layer flat-panel detector (TL-FPD) that comprises at least three energy channels; and, separating the image data into at least three component materials that comprise calcium, water, and contrast agent.

Clause 20: The computer readable media of Clause 19, wherein the contrast agent comprises an iodinated contrast agent.

Clause 21: The computer readable media of Clause 19 or Clause 20, wherein the image data comprises radiographic and/or fluoroscopic image data.

Clause 22: The computer readable media of any one of the preceding Clauses 19-21, wherein the image data comprises interventional radiographic image data.

Clause 23: The computer readable media of any one of the preceding Clauses 19-22, wherein the electronic processor separates the image data into the at least three component materials that comprise calcium, water, and contrast agent using at least one processing technique.

Clause 24: The computer readable media of any one of the preceding Clauses 19-23, wherein the processing technique comprises a polynomial-based technique.

Clause 25: The computer readable media of any one of the preceding Clauses 19-24, wherein the processing technique comprises a model-based technique.

Clause 26: The computer readable media of any one of the preceding Clauses 19-25, wherein the processing technique comprises a machine learning technique.

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

1. A method of decomposing image data obtained from a subject using a computer, the method comprising:
receiving, by the computer, the image data from a triple-layer flat-panel detector (TL-FPD) that comprises at least three energy channels; and,
separating, by the computer, the image data into at least three component materials that comprise calcium, water, and contrast agent, thereby decomposing the image data obtained from the subject using the computer.

2. The method of claim 1, wherein the contrast agent comprises an iodinated contrast agent.

3. The method of claim 1, wherein the image data comprises radiographic and/or fluoroscopic image data.

4. The method of claim 1, wherein the method is substantially insensitive to motion of and/or in the subject.

5. The method of claim 1, wherein the method provides direct visualization of any of the three energy channels.

6. The method of claim 1, wherein the image data comprises interventional radiographic image data.

7. The method of claim 1, wherein the separating step comprises using at least one processing technique.

8. The method of claim 7, wherein the processing technique comprises a polynomial-based technique.

9. The method of claim 7, wherein the processing technique comprises a model-based technique.

10. The method of claim 7, wherein the processing technique comprises a machine learning technique.

11. A system for decomposing image data obtained from a subject, the system comprising:
a processor; and
a memory communicatively coupled to the processor, the memory storing instructions which, when executed on the processor, perform operations comprising:
receiving the image data from a triple-layer flat-panel detector (TL-FPD) that comprises at least three energy channels; and,
separating the image data into at least three component materials that comprise calcium, water, and contrast agent.

12. The system of claim 11, wherein the contrast agent comprises an iodinated contrast agent.

13. The system of claim 11, wherein the image data comprises radiographic and/or fluoroscopic image data.

14. The system of claim 11, wherein the image data comprises interventional radiographic image data.

15. The system of claim 11, wherein the processor separates the image data into the at least three component materials that comprise calcium, water, and contrast agent using at least one processing technique.

16. The system of claim 15, wherein the processing technique comprises a polynomial-based technique.

17. The system of claim 15, wherein the processing technique comprises a model-based technique.

18. The system of claim 15, wherein the processing technique comprises a machine learning technique.

19. A computer readable media comprising non-transitory computer executable instructions which, when executed by at least one electronic processor, perform at least:
receiving image data from a triple-layer flat-panel detector (TL-FPD) that comprises at least three energy channels; and,
separating the image data into at least three component materials that comprise calcium, water, and contrast agent.

20. The computer readable media of claim 19, wherein the contrast agent comprises an iodinated contrast agent.

21.-26. (canceled)
